(19) United States
(12) Patent Application Publication
(10) Pub. No.: US 2026/0325766 A1
(43) Pub. Date: Oct. 22, 2026

(54) HAPTIC FEEDBACK FLOW RATE CONTROL VALVE

(71) Applicant: HOLLO MEDICAL INC., Halifax (CA)

(72) Inventors: David Mark Douglas HODGSON, Halifax (CA); Sara FEDULLO, Halifax (CA); Brandon Alexander BEACH, Ellershouse (CA)

(21) Appl. No.: 19/110,336

(22) PCT Filed: Sep. 8, 2023
(86) PCT No.: PCT/CA2023/051198

§ 371 Date: Mar. 10, 2025

Related U.S. Application Data
(60) Provisional application No. 63/405,551, filed on Sep. 12, 2022.

(51) Int. Cl. A61M 15/00; F16K 15/16; F16K 37/00

(52) U.S. Cl. CPC A61M 15/0015; A61M 15/0018; A61M 15/002; A61M 2205/582; F16K 15/16; F16K 37/0008

(57) ABSTRACT

A flow rate control valve comprising a flexible closure. The flexible closure moveable between an open position and a vibration position. The vibration position providing haptic feedback to a user to signal an inspiratory flow rate above a desired flow rate. The flow rate control valve may be used in connection with a pressurized metered dose inhaler or spacer to provide, for example, longer, slower, and consistently controlled inspiration by flow restriction and flow power conversion into vibratory motion.

### FIELD

The present invention relates to flow rate control valves and more specifically to flow rate control valves with a haptic feedback mechanism.

### BACKGROUND

The following paragraphs are not an admission that anything discussed in them is prior art or part of the knowledge of persons skilled in the art.

Asthma, chronic pulmonary disease and other respiratory illnesses may be treated using medicaments delivered by pressurized metered dose inhalers (PMDI). For example, 1 in 12 children suffer from asthma and make use of PMDIs. An example inhaler may comprise, for example, a plastic holder forming an inlet for a drug canister and a mouthpiece. The drug canister may comprise, for example a propellant with a drug suspension, and a metering valve that is connected to a nozzle for expelling the aerosol drug from the cannister. The cannister may be inserted into the inlet of the plastic holder such that the nozzle of the cannister aligns with the mouthpiece of the plastic holder. When the cannister is actuated, a drug aerosol is released through the mouthpiece. These inhalers may be used on their own whereby a user forms a seal around the mouthpiece of the inhaler and inhales simultaneously with actuating the inhaler. Alternatively, the inhaler mouthpiece may be connected to an aerosol holding chamber (also called a spacer) whereby the user forms a seal around a mouthpiece of the spacer. Facemasks that cover the nose and mouth are also available for use with the spacer and may be prescribed for users that cannot form an airtight seal on a mouthpiece of the spacer or inhaler.

The spacer may be used to contain the high-speed dose particles that are expelled from an inhaler when a user actuates the inhaler. This makes dose inhalation independent of actuation and allows a user to inhale the dose particles at their own rate, for example by dividing the dose over multiple breaths. Spacers further enable the formation of smaller aerosols which increase lung deposition and simultaneously reduces local side effects in the mouth and throat.

### INTRODUCTION

The following introduction is intended to introduce the reader to this specification but not to define any invention. One or more inventions may reside in a combination or sub-combination of the apparatus elements or method steps described below or in other parts of this document. The inventors do not waive or disclaim their rights to any invention or inventions disclosed in this specification merely by not describing such other invention or inventions in the claims.

Inhalers and spacers available on the market are often performance tested at a flow rate of 28.3 LPM. Accordingly, these spacers and inhalers assume a user's inhalation flow rate is at or about 28.3 LPM. In practice however, inherent inspiratory flow is much greater than 28.3 LPM and typically rises to more than 60 LPM. Faster inspiration by a user reduces the time available for the dose to dwell in the lungs and results in undesirable amounts of the dose impacting areas other than the lungs, for example in the throat and mouth.

Flow rate indicating valves that include reed or whistle feedback mechanisms may be used to signal a user to reduce their inhalation flow rate. However, such mechanisms may be confusing or impractical for some environments and users. For example, the reed and whistle feedback may not be practical in loud environments or for users with auditory or visual impairment. The reed and whistle feedback mechanisms further do not prevent misuse by excessive inhalation flow rate and only indirectly measure flow through the valve into the patient's mouth.

A flow rate control valve according to the present disclosure may be used to encourage users inhale at a desired flow rate or within a range of desired flow rates. A desirable inspiratory flow rate for an inhaler may be less than about 60 LPM, less than about 30 LPM, or less than about 15 LPM. A flow rate above a desirable threshold may reduce distal lung dose delivery by about 150% to about 300%, and may cause the dose to impact areas other than the lungs, for example the mouth and throat.

A flow rate control valve according to the present disclosure is provided that may achieve flow control to a desired range through conversion of flow energy into a flexible closure resonant vibration. The flow rate control valve may be used in connection with a spacer and/or an inhaler to prompt a user to adjust their inhalation flow rate to within an optimal range.

A flow rate control valve according to the present disclosure may be used in connection with a spacer or inhaler to provide haptic feedback to users to control flow rate. For example, the haptic feedback or vibration may provide a warning signal that the user's inspiration flow rate is too high and may prompt the user to correct their inhalation flow rate to within a range where the vibration ceases.

A flow rate control valve according to the present disclosure may help encourage patients to maintain inhalation at a flow rate such as to result in longer, slower and more consistently controlled inspiration. The flow rate control valve may further provide an additional mechanism for mechanically restricting flow if the user's inhalation exceeds an upper limit.

A flow rate control valve according to an aspect of this disclosure includes a housing defining a cross-sectional area for fluid flow, and a flexible closure that includes a first end connected to the housing and a second end biased in an open position and moveable between i) the open position, and ii) a vibration position. Fluid flowing through the housing moves the second end from the open position to the vibration position when the fluid flow rate is at a vibration-inducing flow rate.

In another aspect, the present disclosure provides a housing defining a cross-sectional area for fluid flow, and, a flap connected to the housing and comprising a spring constant of from about 25 N/m to about 650 N/m. A fluid flow rate from about 15 LPM to about 60 LPM is sufficient to induce the flap to vibrate.

### BRIEF DESCRIPTION OF THE FIGURES

FIGS. 1A and 1B illustrate perspective views of example flow rate control valves according to embodiments of this disclosure.

FIG. 2 illustrate a front view of an example flow rate control valve according to embodiments of this disclosure.

FIG. 3A illustrates a flow rate control valve with a flexible closure in an open position according to embodiments of this disclosure.

FIG. 3B illustrates a flow rate control valve with a flexible closure in a vibration position according to embodiments of this disclosure.

FIG. 3C illustrates a flow rate control valve with a flexible closure in a closed position according to embodiments of this disclosure.

FIG. 4 illustrates a flow rate control valve with a by-pass control aperture according to embodiments of this disclosure.

FIG. 5 is a graph comparing a traditional prior art valve and a flow rate control valve according to embodiments of this disclosure, under the same inspiratory power.

FIG. 6 is a graph displaying a flexible closure spring characterization relative to vibration onset flowrate.

FIG. 7A illustrates an open clamshell collapsible chamber design with an integrated flow rate control valve according to embodiments of this disclosure.

FIG. 7B illustrates a closed clamshell collapsible chamber design with an integrated flow rate control valve according to embodiments of this disclosure.

FIG. 8A illustrates an exemplary holding chamber in an un-collapsed open position.

FIG. 8B illustrates the exemplary holding chamber in a collapsed position, where the chamber is collapsed perpendicular to the airflow.

FIG. 8C illustrates the exemplary holding chamber in a collapsed position, where the chamber is collapsed parallel to the airflow.

FIG. 9A illustrates a device according to embodiments as disclosed herein comprising a chamber with expendable bellows in a non-expanded position.

FIG. 9B illustrates a top half of a device as disclosed herein comprising a chamber with expanded bellows.

FIG. 10A illustrates an open inhaler port on an expandable bellow chamber, in the expanded position, according to embodiments described herein.

FIG. 10B illustrates the inhaler port of FIG. 9A in the closer position.

FIG. 11 illustrates a cross-sectional side view of an inhaler port with and without an inhaler, wherein the port is on a device with expandable bellows as described in this disclosure.

FIG. 12A illustrates a nasal occlusion face mask according to embodiments disclosed herein.

FIG. 12B illustrates a nasal occlusion face mask associated with a flow rate control valve and collapsible bellows chamber according to embodiments disclosed herein.

FIG. 13 illustrates a ratchet style height adjustment for a nasal occlusion face mark according to embodiments disclosed herein.

FIG. 14A illustrates an example flow rate control valve connected to an inhaler and a holding chamber according to embodiments of this disclosure.

FIG. 14B illustrates an example flow rate control valve connected to an inhaler according to embodiments of this disclosure.

FIG. 15 illustrate a perspective and cross-sectional view of an example flow rate control valve according to embodiments of this disclosure.

### DETAILED DESCRIPTION

In one aspect, the present disclosure provides a flow rate control valve that includes a housing defining a cross-sectional area for fluid flow, and a flexible closure that includes a first end connected to the housing and a second end biased in an open position and moveable between i) the open position, and ii) a vibration position. Fluid flowing through the housing moves the second end from the open position to the vibration position when the fluid flow rate is at a vibration-inducing flow rate. In the examples disclosed below, fluid as discussed herein comprises gaseous fluid, for example gases and aerosols.

The flexible closure may be integrally formed with a housing or formed separately and attached to a housing at one end. The flexible closure may be made of a flexible material, such as for example plastic or silicone. In a further example, the flexible closure may be made of a rigid material but constructed such that it is thin enough to provide the desired flexibility and vibration. For example, the flexible closure may comprise a rigid plate, a conventional hinge, and an externally attached spring tuned to achieve the desired vibration.

The flexible closure may function similar to a spring. For example, the flexible closure may be sized and shaped to have a spring constant of from about 25 N/m to about 650 N/m, for example about 50 N/m to about 500 N/m, or from about 100 N/m to about 300 N/m, for example from about 150 N/m to 250 N/m, or from about 160 N/m to about 200 N/m. The spring constant k of the flexible closure may be determined for example by the thickness, length and elastic modulus of the material of the flexible closure. The spring constant may be used to dictate the balance of forces needed between the inspiratory pressure and the balancing force of the flexible closure such that the flexible closure resonates in phase with the pressure forces acting against it. The spring constant may be measured at or near an end of the flexible closure, for example at the part of the flexible closure furthest away from the connection point of the flexible closure and the housing. In other examples an even smaller spring constant may be needed, for example a spring constant of about 5 N/m may be sufficient when the surface area of the flexible closure is small. Similarly, a larger surface area may require a larger spring constant to achieve vibration of the flexible closure at a desired flow rate.

A first end of the flexible closure may be thicker than a second end of the flexible closure. The first end may be located for example at the part of the flexible closure closest to the connection point of the flexible closure and the housing, and the second end may be located for example at the part of the flexible closure furthest away from the connection point of the flexible closure and the housing. In an example, the flexible closure may comprise a pie-shaped cross-section, such that the first end is thicker than the second end, or such that the second end tapers to a point from the first end. In an example, the second end may be about 30% to about 90% the thickness of the first end, or the second end may be about 50% to about 80% the thickness of the first end, or the second end may be about 70% the thickness of the first end. In another example, the flexible closure may have a uniform thickness. The flexible closure may have a symmetric or asymmetric profile. In some examples, the flexible closure may comprise a uniform thickness up until a specified position and then flow into a tapered second end. In other examples, the flexible closure may have a tapering body from a first end towards a second end, and a further tapered tip that begins a distance from the end of the second end.

A tapered second end, or tapered tip, may help provide an improved seal when the flexible closure is in a closed position as the tapered end more easily abuts against an orifice plate or a contact surface, as further discussed in this disclosure. In an example, a second end before the start of a tapered tip may be a distance from the orifice plate that is equal to a thickness of a first end of the flexible closure. A tapered flexible closure (with or without an additional tapered tip) may form an angle with an adjacent orifice plate. A larger angle may result in a greater inspiratory flow rate before a vibration inducing flow rate is reached. A thicker flexible closure may similarly increase the flow rate required to reach a vibration inducing flow rate. Other flexible closure constructions may be used, for example using traditional style check valve designs such as an umbrella style closure or a duckbill style closure, modified such that a gap exists between the closure and a contact surface resulting in the closure being in an open position at rest. The existence of a gap, contrary to traditional check valves that begin in a closed position, may allow a resonance to occur between the closure ends and the contact surface by enabling the conversion of fluid kinetic energy into resonant vibration of the valve periphery.

The flexible closure may be for example, a flap that extends from the housing from a first end, for example a cantilevered flap, or may be separately attached to the housing, for example using a hinge or other means. In one aspect, the present disclosure provides a housing defining a cross-sectional area for fluid flow, and, a flap connected to the housing and comprising a spring constant of from about 25 N/m to about 650 N/m. A fluid flow rate from about 15 LPM to about 60 LPM is sufficient to induce the flap to vibrate.

The flexible closure may be a single flexible closure or multiple flexible closures. For example, two or three flexible closures may be formed with or attached to a housing. The limit of the number of flexible closures used may be determined by manufacturing limitations and space in the housing.

Multiple flexible closures may provide for haptic feedback at different flow rates. For example, in an embodiment with two flexible closures, the two flexible closures may be configured to have different trigger set points. A first flexible closure may be configured to trigger at 10 LPM, for example, to indicate that a user is inhaling at an acceptable flow rate, while a second flexible closure may be configures to trigger at 60 LPM, for example, to indicate that the user is inhaling at a flow rate that is too high.

When more than one flexible closure is used, for example when two flexible closures are used, a middle column between the flexible closures may be included to split the orifice plate in half such as to form two orifices, each orifice aligned with each of the flexible closures. A small gap may exist between each flexible closure and the orifice plate, for example between the flexible closure and the middle column, when the flexible closures are in a closed position. The small gap may be helpful to provide a path of least resistance, and the most direct flow path, for drug particles through the flow rate control valve which aligns with the location where the highest density, for example the highest number of drug particles, exit the aerosol device (i.e. inhaler). In a single flexible closure embodiment for example, the highest impact density area of drug particles when a user inhales is the center of the valve. However, with a single flexible closure, this highest density of particles would contact the center of the flexible closure and the particles could lose speed before reaching the user's lungs. In a multiple flexible closure environment, for example with two flexible closures, the highest density of particles may benefit from a path of least resistance through the gaps formed between each of the flexible closures and the middle column. As such, an optimized path of least resistance may be formed for the drug delivery by maintaining gaps between the flexible closures and the orifice plate, including between the flexible closures and the middle column (or columns).

Multiple flexible closures covering the area of a single flexible closure may further benefit from comprising a more flexible base compared to a single flexible closure with the same dimensional constraints. In particular, the narrower flexible closures comprise a relatively longer moment arm providing a higher degree of flexibility. The higher moment arm allows the flexible closure to react to more limited fluid flow rates as compared to the lower moment arm for a single flexible closure.

The housing of a flow rate control valve according to any aspect in this disclosure may be formed in a single piece or multiple pieces. In use, the housing may form an air tight or virtually air tight seal around the flexible closure. For example, air or aerosols that enter the housing may not escape the housing other than by passing past the flexible closure or through an optional fail safe or by-pass control, as further described in this disclosure. Preferably, the flow rate control valve, including the flexible closure and housing, is made entirely of an anti-static material in order to minimize aerosol attraction and adherence to the valve.

The cross-sectional area for fluid flow defined by the housing may define a flow path connecting a user interface, for example a mouthpiece for the user, to an inhaler opening or port, such that in use, a dose may flow from the inhaler to the user's mouth. The housing may be connected to or integral with a spacer or inhaler. The cross-sectional area may be further narrowed by one or more contact surfaces forming an orifice plate within the housing. The contact surfaces may be proximate to the flexible closure in the housing, such as to form a seal when the flexible closure abuts the contact surfaces. The contact surfaces may be for example upstream or downstream in the direction of inhalation from the flexible closure. The orifice plate or contact surfaces may define an open area for fluid flow downstream or upstream of the flexible closure. A seal between the orifice plate or contact surfaces and the flexible closure may be formed to substantially close the open area for fluid flow. For example, a seal may be formed when no fluid, or virtually no fluid, for example less than 10% of fluid, is able to pass through the cross sectional area (not accounting for any by-pass control). Throughout this disclosure, “orifice plate” and “contact surfaces” may be used interchangeably to describe the contact surface against which the flexible closure abuts when in a closed position.

The flow rate control valve according to this disclosure may be used to control a user's inspiratory flow rate using a haptic or vibrational feedback system. For example, the flow rate control valve may be used to encourage a user to inhale at a predetermined flow rate or range of flow rates using, for example, biofeedback and muscle training. In an example, the predetermined flow rate may be below a vibration inducing flow rate such that when a user's inspiratory flow rate is above a vibration inducing flowrate the valve vibrates, providing the user with a mechanical indication that their inspiratory flow rate is above a desired threshold and prompts the user to lower their inhalation rate. Once a user lowers their inhalation rate below the vibration inducing flowrate, the vibrations cease. In another example, the predetermined flow rate or range of flow rates may be at or above the vibration inducing flow rate such that when a user's inspiratory flow rate corresponds to a vibration inducing flowrate, the valve vibrates, providing the user with a mechanical indication to maintain their inspiratory flowrate. In a specific example, the flow rate control valve may provide haptic or vibrational feedback when the user's inhalation rate is from about 20 to about 30 LPM, but not when the inhalation rate is outside of this range. The vibration threshold may be for example, from about 15 LPM to about 60 LPM, for example, from about 20 LPM to about 50 LPM, or about 30 LPM.

The neutral or equilibrium position of the flexible closure, when the flexible closure is at rest and there is no inspiratory flow, or the inspiratory flow is below a threshold, for example below about 15 LPM, is the open position. In the open position, fluid such as air, mist or aerosols may flow past the flexible closure through the cross-sectional area without any vibration of the flexible closure. As a user's inspiratory flow increases up to a threshold flow rate, for example a flow rate above a vibration inducing flow rate, for example above about 15 LPM or above about 30 LPM, and up to about 60 LPM, the flexible closure may move into a vibration position. In an example, the flexible closure may begin to move from the open position into the vibration position within about 10% of a vibration inducing flow rate. For example, an inspiratory flow rate of about 10 LPM will result in the flexible closure remaining in the open position or stationary in its starting open position, however as the user's inspiratory flow moves into within 10% of the vibration inducing flow rate, for example 15 LPM, the flexible closure moves into a vibration position. The vibration inducing flow rate may be determined or selected for suitability with the user based on the spring constant of the flexible closure. When in the vibration position, a portion of the kinetic airflow energy is converted to deflect the spring at its resonant frequency. In a vibration position, the flexible closure may extend back and forth, towards the orifice plate without contacting the orifice plate and past equilibrium in the opposite direction. The vibrations of the flexible closure provide haptic feedback to the user. The haptic feedback may trigger a warning or cue for the user that their inspiratory flow rate is in excess and thereby prompts that the user to reduce their inspiratory flow. In an example, the flexible closure may vibrate at a range of frequencies, however, the maximum frequency will be its resonance frequency. The whole functional vibration range however may provide feedback to the user.

When an inspiratory fluid flow rate is significantly above an optimal flow rate, and above a vibration inducing flow rate, for example, if the inspiratory flow rate is above about 60 LPM, the flexible closure biases to the closed position. The closed position may prevent about 90% of fluid from flowing past the closure (not accounting for any by-pass control). The flexible closure may be sized to cover the entire aperture formed by the orifice plate or at least above 90% of the aperture formed by the orifice plate. In the closed position, the flexible closure may abut or seal against the orifice plate to prevent fluid from flowing past the flexible closure.

The flow rate control valve according to this disclosure may include a by-pass control. At least one of the orifice plate, the flexible closure and the housing may define for example, a by-pass aperture for fluid flow when the flexible closure is in the closed position. In another example, the closed position of the flexible closure may form a gap between the housing and the flexible closure sufficient to act as a by-pass aperture. The by-pass aperture may be shaped and sized to limit the fluid flow rate to a specified or optimal flow rate. For example, in the event a user's inhalation far exceeds an optimal inhalation threshold, for example, above a vibration inducing flowrate, such as for example greater than about 60 LPM, the valve may close and force the user to inhale only through the by-pass aperture. Accordingly, if an inspiratory flow rate exceeds a vibration inducing flow rate, and the flexible closure moves into a closed position, a user can nevertheless inhale through the flow rate control valve by means of the by-pass aperture. The by-pass aperture provides a failsafe to restrict the flowrate of the inhaled dose even when the user's inhalation far exceeds a desired rate. The fluid dose from the inhaler in such cases will be forced to move through only the by-pass aperture that is sized and shaped to control the flowrate to a specified amount.

The flow rate control valve according to this disclosure may comprise a check valve. The check valve may be positioned downstream, in the direction of inhalation, from an orifice plate. The check valve may be formed separately and attached to the housing or formed integrally with the housing. For example, when formed separately from the housing, a plate on a conventional hinge with an externally attached spring for example, may be used for the check valve. The check valve may be a flap. At rest or without any inspiratory flow, the check valve is closed. The closed position may be defined as the check valve abutting against the orifice plate to form a seal. In another example, the check valve may be closed by sealing against another separate contact surface other than the orifice plate. Since the check valve is at equilibrium or at rest in the closed position, it does not oscillate or vibrate like the flexible closure. The check valve is either in an open position when the user inspires or a closed position when there is no inspiration flow. The spring constant of the check valve may be tuned such that a user's natural inspiratory flow exceeds the force required to open the valve, quickly or immediately and causes the check valve to open. The check valve may function to ensure that any fluid such as air, mist, or aerosols introduced into the housing from, for example an inhaler, remain in the housing or in an attached chamber, without escaping into the atmosphere when there is no inhalation. The check valve may also function to control the direction of flow and helps prevent a user's exhalation from passing back through the check valve, the flexible closure or into any attached chamber or inhaler. The check valve may be complemented by an exhalation port. In another example, an exhalation port may be present without a check valve. The exhalation port provides a means for expelling a user's exhalation that enters the housing before it flows back past the flexible closure. The exhalation port may be for example formed in the housing proximate to the user's mouthpiece.

Similar to the flexible closure, the check valve may comprise one or multiple check valves. For example, in an embodiment with two check valves, the effort to open the check valve during inhalation may be reduced as compared to a single check valve. For example, a lower flow rate may be capable of opening multiple check valves as compared to a single check valve. In particular, as previously discussed in reference to the flexible closure, a more flexible base may be used for multiple smaller check valves as compared to a single check valve with the same dimensional constraints, and may enhance the moment arm with limited flow rate.

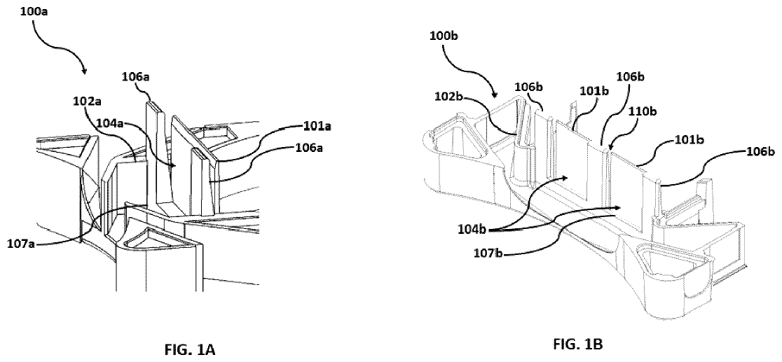

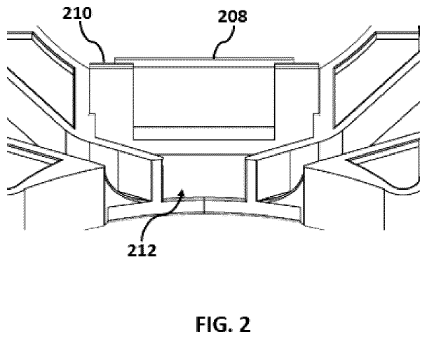

FIGS. 1A, 1B, and 2 illustrate perspective and front views of example flow rate control valves according to embodiments of this disclosure. Each of FIGS. 1A, 1B and 2 shows a housing and one or more flexible closures. However for the purpose of illustration and identification of parts, the upper half of the housing in FIGS. 1A, 1B, and 2 is omitted.

FIG. 1A depicts a flow rate control valve 100a according to an embodiment comprising a single flexible closure 101a. The flow rate control valve comprises a housing 102a defining a cross-sectional area 104a for fluid flow. FIG. 1A shows two vertical contact surfaces 106a and a horizontal contact surface 107a connecting the bottom of the two vertical contact surfaces to form the lower and side portions of an orifice plate. An additional horizontal contact surface connecting the top of the two vertical contact surfaces may be added, for example from an upper half of the housing. Any one or more of the contact surfaces may be omitted in other examples so long as a substantial seal may be formed when the flexible closure is in a closed position (as described herein). For example, the orifice plate may be formed in a single piece with a cut-out or hole defining a narrowed cross-sectional area wherein when the flexible closure is in a closed position, the open area for fluid flow is substantially sealed. In another example, multiple contact surfaces may be connected to one another to form an orifice plate with an open area for fluid flow that is substantially sealed when the flexible closure is in a closed position. A substantial seal as discussed throughout this disclosure indicates that no fluid or no more than 5% of the volume of fluid passes through the seal (without accounting for any by-pass control).

FIG. 1B depicts a flow rate control valve 100b according to an embodiment comprising two flexible closures 101b. The flow rate control valve comprises a housing 102b defining two cross-sectional areas 104b for fluid flow. FIG. 1B shows three vertical contact surfaces 106b and a horizontal contact surface 107b connecting the bottom of the three vertical contact surfaces to form the lower, side and middle column portions of an orifice plate. An additional horizontal contact surface connecting the top of the three vertical contact surfaces may be added, for example from an upper half of the housing. Any one or more of the contact surfaces may be omitted or added in other examples, such as when more or less than two flexible closures are used. When more than one flexible closure is sealed, the closed position of each of the flexible closures may allow for a gap for fluid to flow between the side edges of the flexible closures and the vertical contact surfaces of the orifice plate. For example, a gap 110b may be formed between the side edges of each of the flexible closures and the middle column of the orifice plate. The orifice plate may be formed in a single piece with cut-out or holes defining narrowed cross-sectional areas wherein when the flexible closures are in a closed position, the open area for fluid flow is substantially sealed other than the small gaps formed between the vertical contact surfaces and the edges of the flexible closures. In another example, multiple contact surfaces may be connected to one another to form the orifice plate with open areas for fluid flow that is substantially sealed except for the small gaps as described above, when the flexible closure is in a closed position. The small gap may be helpful to provide a pathway of least resistance for the greatest proportion of drug particles since the highest impact density area of drug particles when a user inhales is the center of the valve. For example, with two flexible closures, the highest density of particles may benefit from a path of least resistance through the small gaps formed between each of the flexible closures and the middle column. As such, an optimized path of least resistance may be formed for the drug delivery by maintaining these small gaps between the flexible closures and the orifice plate, including between the flexible closures and the vertical contact surfaces.

FIG. 2 illustrates a front view of the flow rate control valve according to an example as disclosed herein with a single flexible closure. Other embodiments may include 2 or more flexible closures. The orifice plate 210 and flexible closure 208 are positioned proximate to a mouthpiece 212 (only aperture for mouthpiece pictured). In a further example, a check valve may be positioned between the mouthpiece 212 and the orifice plate 210. Two or more check valves may be used in other embodiments. In addition, the flow rate control valve according to any embodiment as described herein, may further include a by-pass control, for example a by-pass aperture in the orifice plate, in the one or more flexible closures or in the housing. In a further example, an aperture sufficient to act as a by-pass control may be alternatively or additionally formed by the flexible closure and the orifice plate when the flexible closure is in the closed position, for example, as a small gap between the flexible closure and the orifice plate.

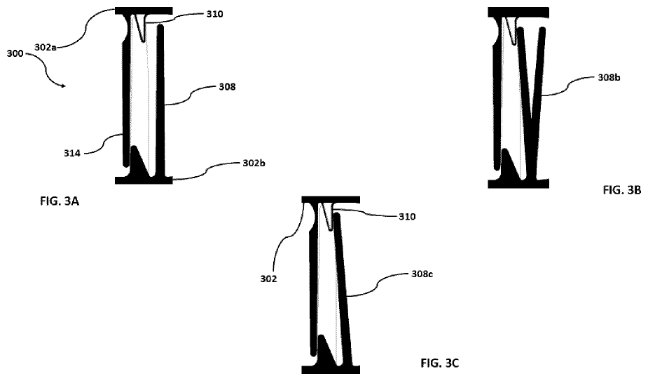

FIGS. 3A-3C provide an example flow rate control valve 300 in various positions. The flow rate control valve according to this embodiment comprises a flexible closure 308 upstream (in the direction of inspiratory flow) of an orifice plate 310. The flow rate control valve as shown in FIGS. 3A-3C also includes a check valve 314 positioned downstream of the orifice plate such that the orifice plate 310 is positioned between the flexible closure 308 and the check valve 314. In another example, the flexible closure may be positioned downstream of the orifice plate. The flexible closure as shown in FIGS. 3A-3C tapers about 30% from a first end towards a second end and then further tapers to a tip that is about 40% the thickness of the first end. The flexible closure abuts the orifice plate 310 at a first end and extends away from the orifice plate a distance at the second end, when in the open position, as shown in FIG. 3A.

The flexible closure 308a as shown in FIG. 3A starts in an open position when the flow rate control valve is not in use or when a user's inspiratory flow is below a vibration inducing flow rate. As a user's inspiratory flow increases up to a vibration flow rate, for example when the user's inhalation flow rate is above about 15 LPM or above about 30 LPM, and up to about 60 LPM, the flexible closure 308b moves into a vibration position as shown in FIG. 3B. The flexible closure oscillates about its equilibrium (or open) position by moving towards the orifice plate without contacting the orifice plate and in the opposite direction past equilibrium. The oscillation causes mechanical vibrations of the flow rate control valve which may be used to indicate to a user that their inhalation is above a desired inhalation flowrate. In an example, the flexible closure may begin to move from the open position into the vibration position within about 10% of the vibration inducing flow rate. The vibrations remind or prompt the user to reduce their inhalation flow rate. If the user increases their flow rate substantially above the vibration inducing flow rate, for example above about 60 LPM, the flexible closure moves to a closed position by contacting and sealing against the orifice plate. FIG. 3C depicts a flow rate control valve with a flexible closure 308c in the closed position. In a closed position, the flexible closure substantially prevents fluid from flowing through the housing 302, past the closure.

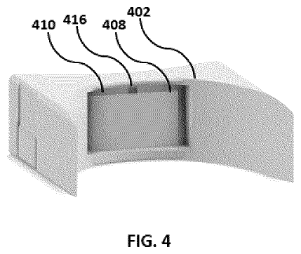

In an example as shown in FIG. 4, the flow rate control valve includes a by-pass control aperture 416. The by-pass control aperture 416 is formed in the orifice plate 410 in FIG. 4, however in other examples the by-pass control aperture may be formed in the housing 402, or in the flexible closure 408. In another additional or alternative example, the closed position of the flow rate control valve may form a gap between the housing and the flexible closure sufficient to act as a by-pass control aperture. The by-pass aperture is sized and shaped to limit the fluid flow to a specific or optimal flow rate, such as for example less than about 40 LPM, for example about 20 LPM or for example about 10 LPM.

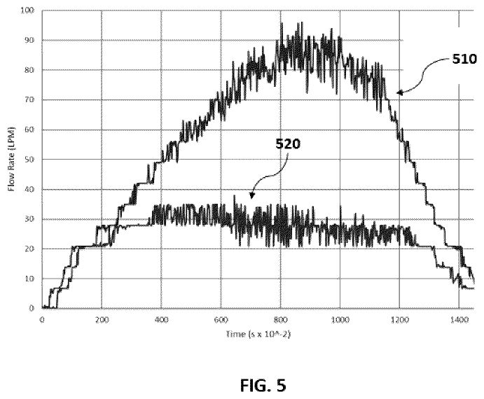

FIG. 5 is a graph comparing a traditional prior art valve 510 and a flow rate control valve 520 according to embodiments as disclosed herein, under the same inspiratory power. Up to a prescribed flow set point, the flow rate control valve according to this example remained in an open position without increasing inspiratory effort (see lower line), it then moved into a vibration position when a user's inhalation increased to about 30 LPM. The vibrations caused the user to reduce their inhalation flow to the optimal rate based on the perceptible vibratory feedback to the user. If the flow dramatically exceeded the vibration inducing flow rate, the flexible closure would have move to the closed position, signaling to the user to further adjust their inspiration, or where a by-pass control aperture is present, could maintain an optimal flow by restricting the flow rate through the aperture. The prior art device in this case is an AeroChamber® by Trudell Medical International which comprises a chamber but no flow control. The AeroChamber® allowed for inspiratory flow well above an optimal flow threshold as the user's inherent inspiratory flow was much greater than the desired flow for metered dose inhalation. It is suggested that the AeroChamber® may result in poor distal lung dose delivery and impaction in areas other than the lungs, for example the mouth and throat, because the user's inhalation is above a desired flow rate.

An exemplary flow rate control valve according to this disclosure was constructed with a flexible closure comprising a material with an 80 MPa elastic modulus, an area of 472 mm\hat{ }2 and a thickness as depicted in FIG. 3A-C. Exemplary spring constants and related vibration inducing flow rates are depicted in Table 1.

TABLE 1
Spring characterization relative to vibration flow rate

| | | | Spring | Vibration |
|---|---|---|---|---|
| Flap | Force | Deflection @ | Constant/k | Inducing |
| (area 472 mm\hat{ }2) | (N) | Tip (mm) | (N/m) | Flowrate (LPM) |
| 1 | 1.34 | 5 | 268 | 37 |
| 2 | 0.83 | 5 | 166 | 31 |
| 3 | 0.54 | 5 | 108 | 28 |

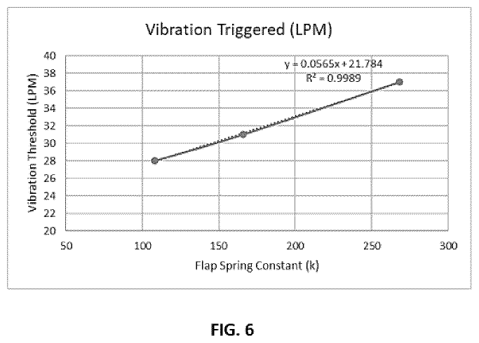

Using the above table data, an example relationship of the spring constant k and the onset of vibration (y) were graphed as shown in FIG. 6. A linear correlation of y=0.0565k+21.78 was determined from this data. From this correlation, it was possible to tune the flexible closure to vibrate once it was contacted with fluid at the desired vibration inducing flow rate. The flexible closure may additionally or alternatively be varied dimensionally and materially, within practical limitations of manufacturing and usability, to achieve the same vibration inducing flow rate and function, or adjust the vibration inducing flow rate accordingly.

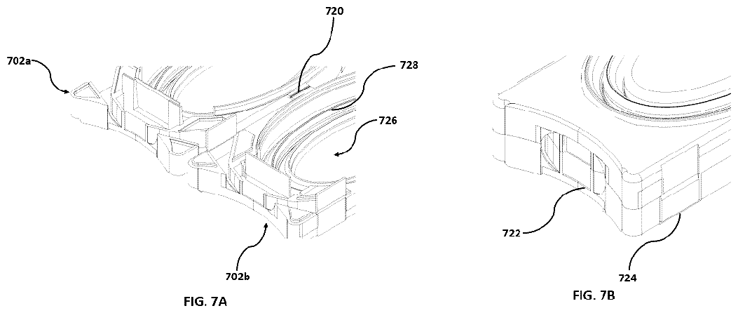

In another example, the flow rate control valve according to any one of the embodiments described in this disclosure may be constructed with an integrally attached spacer or aerosol chamber. The device may be constructed in a single piece or multiple pieces, for example, the device may be formed in as few as a single injection molded part. FIGS. 7A and 7B illustrate an example clamshell design where the chamber and flow rate control valve are integrally formed from a single material. FIG. 7A illustrates the clam shell design in an open configuration, wherein the two halves of the device are connected by a living hinge 720. The check valve in this example is integral with a first half 702a, while the flexible closure is integral with a second half 702b. The flow rate control valve may be constructed by the union of the first half and the second half by folding the shell design in half over the living hinge 720. FIG. 7B illustrates the closed design. The flow rate control valve is proximate to the user interface mouthpiece 722. An exhalation port 724 is also included in this example. The exhalation port allows a user to exhale without removing the device from their mouth. During exhalation, the exhalation port opens to expel air while the check valve remains closed to prevent exhalation air from entering the chamber 726.

Known prior art holding chambers and spacers may require large volumes to maximize clinical performance for example to achieve better bronchial distribution due to the formation of a more respirable dose. The large volumes make these spacers cumbersome and impractical for travel and everyday use. In an example, the flow rate control valve according to any one of the embodiments described in this disclosure may be constructed with an integrally or separately attached holding chamber comprising a variable volume. For example, the holding chamber's volume may be varied by way of expandable (and collapsible) bellows. The variable volume chamber may provide a compact solution to the cumbersome size problem of prior art holding chambers. For example, a chamber comprising expandable bellows may collapse to make the holding chamber compact enough for portability and travel, while remaining large enough in the expanded position to provide a required tidal volume. The expandable bellows may be partially or fully expanded to account for different tidal volume needs. For example, patients of different ages have differing tidal volumes, meaning they require a different number of breaths to clear a device of a fixed volume. Enabling the chamber to have not just one opened position, but two or more, wherein at least one is for a pediatric or lower tidal volume setting, and another is more suited for adults with larger tidal volumes, would be beneficial for universal usage. For example, in one position the device may be open to about a 100 mL volume, in another it may be open to about a 300 mL volume.

In an example, the chamber may be expandable and collapsible in a direction parallel to a fluid flow path such as a chamber with expandable bellows arranged such that the bellows expand in the direction parallel to the fluid flow path. When the chamber volume varies by expanding or collapsing the chamber parallel to the direction of the fluid flow path, the distance between an inhaler port and a mouthpiece may increase or decrease based on the expansion or collapse of the chamber. In another example the chamber may be expanded or collapsed in a direction that is perpendicular to the fluid flow path, such as a chamber with expandable bellows arranged such that the bellows expand in a direction perpendicular to the direction of the fluid flow path. When the chamber volume varies by expanding or collapsing the chamber perpendicular to the direction of the fluid flow path, the distance between an inhaler port and a mouthpiece remains relatively unchanged. Varying the volume of the chamber, for example by arranging the expandable bellows, in a direction perpendicular to the direction of the fluid flow path may reduce aerosol impaction as compared to holding chambers with expandable bellows parallel to the fluid flow path. In particular, geometry that folds parallel to the airflow shortens the distance between the aerosol source (for example an MDI) and the opposing side of the chamber, which may have an adverse effect on the amount of medication that is deposited on the inside of the chamber.

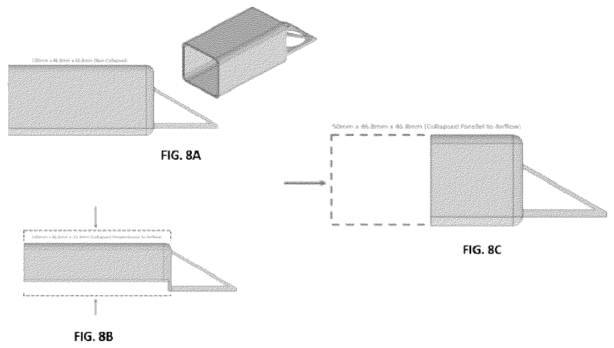

In an example experiment conducted to compare non-collapsed bellows (1), bellows collapsed perpendicular to airflow (2) and bellows collapsed parallel to airflow (3), the inventors found that the chamber that collapsed parallel to airflow had a 13% increase in aerosol impaction as compared to the chamber that collapsed perpendicular to airflow at the same tidal volume. The experiment was conducted using aerosolized deodorant as an analogue to represent aerosolized medication from a metered dose inhaler. The experimental set up consisted of three chambers which were 3D printed using stereolithography technology. The 3D printed chambers represented a holding chamber in an un-collapsed open position (FIG. 8A), and two chambers collapsed to 50% volume of the non-collapsed chamber. The latter were created as follows: the first represented a chamber collapsed perpendicular to airflow (FIG. 8B), and the second represented a chamber collapsed parallel to airflow (FIG. 8C). Each chamber was placed on top of an aluminum-based deposition sheet, intended to collect the aerosolized particles at impaction. The aerosolized deodorant was sprayed through the three chambers and at completion, each element of the experimental set up was weighed to determine the total mass that had been collected inside the chambers versus the spray that impacted the deposition sheet. Summary results of the experiment are shown below in Table 2 below.

TABLE 2
Summary table of results from dry particle deposition testing

| | | | Chamber | Chamber | Spray | Spray | |
|---|---|---|---|---|---|---|---|
| | | | Dry | Wet | Captured | Captured | Total % |
| Chamber | Dimension | Volume | Mass | Mass | in Chamber | on Plate | of Spray |
| Unit # | (cm) | (cm³) | (g) | (g) | (g) | (g) | Captured |
| 1 (non- | 10 × 4.68 × | 219 | 54.9 | 56 | 1.1 | 1.2 | 47% |
| collapsed) | 4.68 | | | | | | |
| 2 (collapsed | 10 × 4.68 × | 109.5 | 45.1 | 46.5 | 1.4 | 0.8 | 43% |
| perpendicular | 2.34 | | | | | | |
| to airflow) | | | | | | | |
| 3 (collapsed | 5 × 4.68 × | 109.5 | 31.6 | 32.9 | 1.3 | 1.5 | 56% |
| parallel to | 4.68 | | | | | | |
| airflow) | | | | | | | |

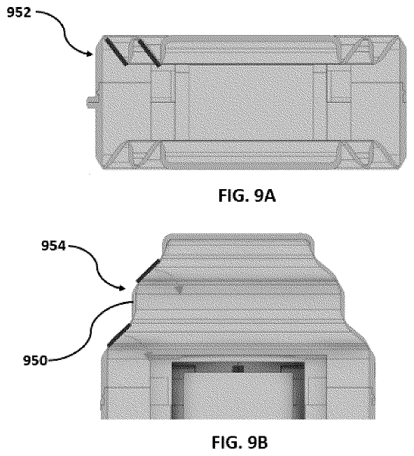

FIGS. 9A and 9B illustrate an example of expandable bellows that expand in a direction that is perpendicular to the fluid flow path. FIG. 9A illustrates the expandable bellows 950 in a closed or non-expanded position 952. FIG. 9B illustrates a top half of the device with the bellows 950 in the expanded position 954 (bottom half omitted but may be symmetrically expanded or asymmetrical expanded). A truncated cone or funnel shape of the expanded expandable bellows in a direction perpendicular to the flow of fluid, for example as shown in FIG. 9B, may further provide central dilation to enable particle expansion, and a slow funneling of particles into the mouthpiece. The bellows comprise angled ringed portions connected by living hinges to form the chamber. The angled rings can rotate approximately 90 degrees from a folded or collapsed position to an open or expanded position and function to reduce the size of the whole device by a factor of 5, in this example, when folded compared to when open. A rigid center structure is also provided to produce an outward force to prevent unfolding.

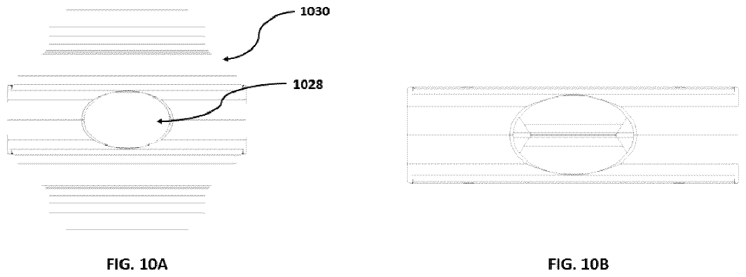

A port for engaging an inhaler to the expandable bellows may be further provided with any of the flow rate control valves as described herein. The inhaler port may be automatically closed when the expandable bellows are in a folded position and opened with inhaler retention features when the bellows are expanded. The inhaler port may be shaped and sized to accept an inhaler or an inhaler housing (for example an inhaler mouthpiece). In an example as shown in FIGS. 10A and 10B, the inhaler port 1028 comprises an oval shape that is actionable by the opening (10A) and closing (10B) of opposing bellows 1030 of a holding chamber. The port in the active or open position, may be open with rib-like projections to allow the insertion and retention of an aerosol generating device, and in the closed position, may be entirely sealed through the collapse of the device's volume expanding bellows. For example, as shown in FIGS. 10A and 10B, the collapse of opposing bellows is configured to cause the edge of the bellows to rotate about 90 degrees and form a seal with the edge of an opposing below such as to help prevent dirt from entering the port when in the closed position. When the device is expanded and its volume is maximized to allow expanded aerosol containment, the port is simultaneously open as the bellow edges move away from the port hole. When the device is folded shut, the port seals to help prevent debris from collecting and contaminating the chamber, for example, when not in use.

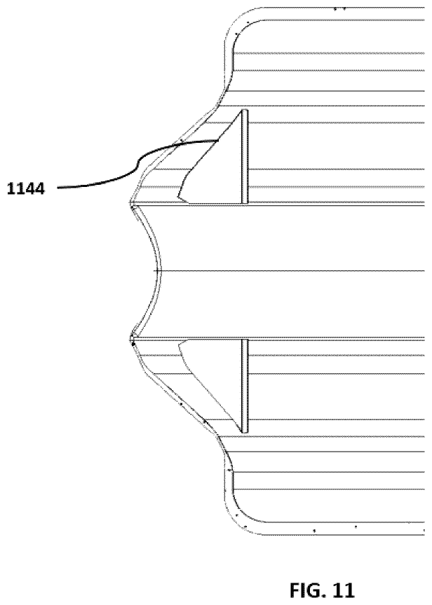

In addition to opening and closing the inhaler port mechanism to prevent dirt and debris entering the device as shown in FIGS. 10A and 10B, the expanding bellows can also facilitate the movement of inhaler retention features in and out of position as shown in FIG. 11. FIG. 11 illustrates a cross-sectional side view of an inhaler port with and without an inhaler, wherein the port is on a device with expandable bellows as described in this disclosure. Additional mechanisms, such as fins shown in FIG. 11 for example, can be added to the expandable bellows adjacent to the inhaler port. By placing, for example, 4 additional fins 1144 (2 top, 2 bottom shown in FIG. 11), it may be possible to cradle the inhaler when it is in its functional position. More or less fins or other retention means may be used in other examples and with any flow rate control valve as described in this disclosure. The inhaler retention features as disclosed herein may enable more robust accommodation and retention of a variety of inhalers with differing mouthpiece profiles. Further, it may also be feasible for the inhaler cradle fins to serve as the inhaler port closure mechanism to prevent dirt and debris from entering the device.

Alternatively, the inhaler port may replace the base of a conventional inhaler “boot” or housing which enables the canister to be actuated directly into the chamber without needing the inhaler housing. The benefit of this, may be particularly seen in hospital settings where the sharing of the same canister across multiple devices/patients may be beneficial. For example, a canister with 120 doses, and an inpatient that only requires acute administration of 8-12 doses, may be able to share the same canister. In the outpatient setting, the canister could feasibly be stored alongside the chamber when not in use, via a standard c-shaped clip.

The flow rate control valve according to any of the embodiments disclosed herein may be constructed with the addition of a nasal occlusion face mask. Presently available holding chamber facemasks known in the prior art rely on a design which forms a triangular seal from the bridge of the nose to the lateral edges of the chin. This is known to be variably effective due to the unpredictable facial anatomy that needs to be accommodated, for being intrusive and frightening to some patients, for increasing the dead space, or air that first needs to be inhaled into the lungs prior to the active drug, resulting in lower efficacy with smaller tidal volumes, and resulting in facial discoloration due to exposure of inhaled corticosteroids to the skin unnecessarily. Facemasks however are intended to enable patients unable to form a seal on a traditional mouthpiece to use a holding chamber and therefore remain an important feature in the aerosol delivery suite of accessories. The nasal occlusion facemask according this disclosure may accommodate all facial anatomies with a single design, utilize only the optimal route of drug inhalation, and may minimize the material wasted as these parts are used in extremely high volumes. There is further the opportunity to use the nasal occlusion design as disclosed herein to customize accommodations for unique facial geometries such as facial deformities, or those with additional medical equipment use (for example, with nasal cannulas in place).

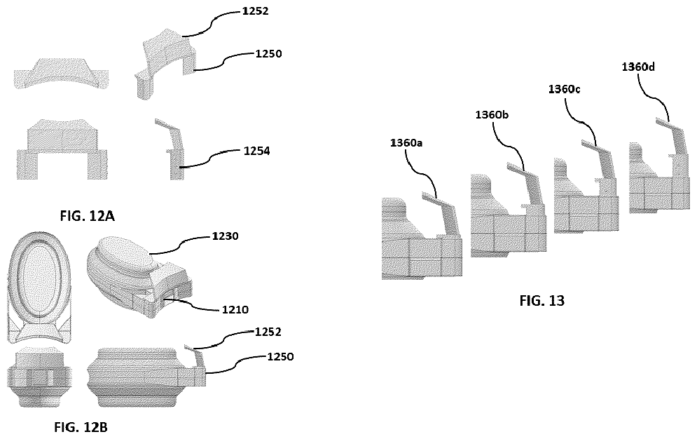

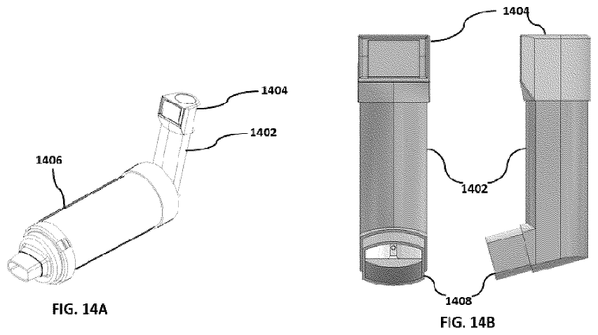

FIG. 12A depicts an example nasal occlusion face mask according to this disclosure which may be used with any flow rate control valve or feature as previously discussed in this disclosure. FIG. 12B depicts an example nasal occlusion face mask associated with a flow rate control valve 1210 and collapsible bellows chamber 1230. The nasal occlusion face mask comprises an over the lip mask 1250, which utilizes the lips as a gasket to create an airtight seal, along with a contoured surface 1252 designed to bilaterally occlude the narus. The mouth portion of the mask may accommodate for example, the 95% ile of mouths, and any variation in height from the mouth to the tip of the nose can be accommodated through an adjustable height, for example ratchet style, of the nasal occlusion surface. This ratchet style adjustment 1254 shown in FIG. 12A is further depicted in FIG. 13 in its various heights. The ratchet style adjustment can be used either manually using height adjustment nuts disposed thereon, or during device use when a seal is formed on the face via a cam-sliding mechanism.

The distance between the base of the nose and the central incisor in developed humans varies between about 19 mm to about 36 mm, while the overall mouth width varies between about 41 mm to about 62 mm. This T-shaped region of human anatomy is predictable and captures the only airway access attainable by masks. By creating a variable height, ratcheting nasal occlusion surface (1360a-1360d) as shown for example in FIG. 13, which can accommodate the anatomical variation of the nose to incisor distance, a broad, c-shaped over the lip mask can replace a mask which commonly covers the chin, mouth, and entire nose. A single device with a ratcheting nasal occlusion plate can supplant the need for various different sizes of facemasks, which require substantial pressure to form a seal over a large surface area that is not involved with gas exchange at all, nor anatomically reliable for a universal fit.

It is additionally noted that the nasal occlusion face mask may be adapted to accommodate atypical facial geometries, for example due to deformity (i.e. cleft lip), or obstructions for example the use of nasal cannulas by way of hospital oxygenation. Moreover, there is the ability to customize the face mask based upon appearance, maintaining function but enabling distractors to certain patient populations (i.e. moustache shaped). For the more custom designs, the use of facial scanning, and personalized 3D printed nasal occlusion masks may also be created. This may enable the ability to not only adapt the nasal occlusion plate, but to enhance the mouth mask fit for those with deformities or obstructions around the mouth.

In a further example, the nasal occlusion face mask is formed in a separate piece as shown in FIG. 12A and attached to a chamber as shown in FIG. 12B. In another example, the nasal occlusion face mask may be integrated with a chamber and deployed with the expansion of the chamber bellows and retracted when not in use. In another example, the contoured surface configured to bilaterally occlude the narus may be constructed of a bendable material such that it may conform to differing anatomies.

FIGS. 14A and 14B show other embodiments of flow rate control valves as disclosed herein. The flow rate control valves as shown in FIGS. 14A and 14B maybe be connected directly to an aerosol producing device, for example to an inhaler 1402, without being in contact with the aerosol dispensed from the inhaler. In an example, as in FIG. 14A, the aerosol producing device or inhaler 1402 may be connected to the flow rate control valve 1404 at an air-inlet end and a spacer or other aerosol or mist-holding chamber 1406 at an aerosol dispensing end. In an example, as in FIG. 14B, a user lip surface 1408 may be on an aerosol dispensing end of the aerosol producing device 1402 with the flow rate control valve 1404 disposed on an air-inlet end.

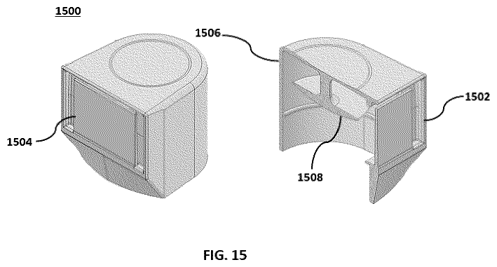

The flow rate control valve of FIGS. 14A and 14B is shown in FIG. 15 as detached from the aerosol producing device. FIG. 15 shows both a perspective and cross-section view of the flow rate control valve. The flow rate control valve 1500 according to this embodiment comprises an orifice plate 1502 and flexible closure 1504 as previously disclosed however, in this embodiment, the orifice plate and flexible closure are disposed upstream (in the direction of inhalation) from the aerosol producing device, for example, at an air inlet end of an inhaler. This configuration may be helpful in controlling a user's inhalation rate without contacting the drug aerosol expelled from the aerosol producing device, which could otherwise contact the flexible closure. Drug delivery may be optimized by avoiding loss of drug particles contacted with the flexible closure. This embodiment may further be helpful as an addition for a device comprising an inhaler with a conventional holding chamber and check valve, and may be added to the top of the inhaler and removed as desired. For example, a user may choose to add the flow rate control valve to the inhaler for training purposes and remove the flow rate control valve when the user is sufficiently comfortable with an acceptable inhalation range.

The flow rate control valve 1500 may comprise a housing 1506 configured to attached to an air inlet end of an aerosol producing device, for example, the housing may be configured to attach to the plastic holder of an inhaler. Inhalers typically include a canister that must be actuated to release the drug aerosol to the user. The flow rate control valve housing may be attached to the air inlet end of the inhaler above the canister. The housing may comprise a flexible diaphragm 1508 that is aligned with the top of the canister such that when a user depresses the flexible diaphragm, an extension of the flexible diaphragm 1508 contacts and actuates the canister to release the drug aerosol to the user through the aerosol dispensing end of the inhaler. The flow rate control valve may comprises one or more flexible closures 1504, such that air inhaled by a user from the air dispensing end of the inhaler enters the housing of the flow rate control valve and thus the air inlet end of the inhaler, via the flexible closure(s).

Multiple flexible closures may be configured as previously described in other embodiments of this disclosure. The flexible closure(s) may be adjusted as described in this disclosure to maintain optimal inhalation rates. For example, as previously discussed in this disclosure, the flow rate control valve may be used to encourage a user to inhale at a predetermined flow rate or range of flow rates using, for example, biofeedback and muscle training.

In an example, the predetermined flow rate may be below a vibration inducing flow rate such that the valve vibrates when a user's inspiratory flow rate is above a vibration inducing flowrate, providing the user with a mechanical indication that their inspiratory flow rate is above a desired threshold and prompting the user to lower their inhalation rate. Once a user lowers their inhalation rate below the vibration inducing flowrate, the vibrations cease. In another example, the predetermined flow rate or range of flow rates may be at or above the vibration inducing flow rate such that the valve vibrates when a user's inspiratory flow rate corresponds to a vibration inducing flowrate, providing the user with a mechanical indication to maintain their inspiratory flowrate. In an example, a user may begin to inhale before actuating the aerosol producing device by depressing the diaphragm, and only actuating the diaphragm when the optimal inhalation flow rate is reached based on the feedback provided by the vibrations of the flexible closure. The vibration trigger points may be selected to account for whether the inhaler is also attached to a holding chamber or if it is to be in direct contact with a user's mouth or with a face mask. For example, inhalers used without a holding chamber need to be inhaled at a higher flow rate as compared to when a holding chamber is used, to ensure that the drug is delivered to the lungs. In such case, the vibration trigger point is selected for the flexible closure to maintain the optimal increased flowrate.

The flow rate control valve according to any of the embodiments in this disclosure may be used as a device for delivering a medicated aerosol or mist to a user. For example, the flow rate control valve may be directly connected to an inhaler (for example, a pressurized metered dose inhaler) or connected to a spacer, such as an expandable bellows spacer, that is connected to an inhaler. In an embodiment with a spacer, the spacer is upstream of the flow rate control valve, and the inhaler is upstream of the spacer, in the direction of inspiratory flow. The device may be used for the treatment or prevention of asthma, chronic obstructive pulmonary disease or any other treatment requiring dose delivery through the pulmonary system.

We claim:

1. A flow rate control valve comprising,
a housing defining a cross-sectional area for fluid flow, and
a flexible closure comprising a first end connected to the housing and a second end biased in an open position and moveable between i) the open position, and ii) a vibration position,
wherein fluid flowing through the housing moves the second end from the open position to the vibration position when the fluid flow rate is at a vibration-inducing flow rate.

2. The flow rate control valve of claim 1 wherein the flexible closure is sized and shaped to have a spring constant of from about 25 N/m to about 650 N/m, for example about 50 N/m to about 500 N/m, or from about 100 N/m to about 300 N/m, for example from about 150 N/m to 250 N/m, or from about 160 N/m to about 200 N/m.

3. The flow rate control valve of claim 1 or 2 wherein the first end of the flexible closure is thicker than the second end of the flexible closure, for example the second end is about 30% to about 90% the thickness of the first end, or the second end is about 50% to about 80% the thickness of the first end, or the second end is about 70% the thickness of the first end.

4. The flow rate control valve of any one of claims 1 to 3 wherein the vibration inducing flow rate is between about 15 LPM and about 60 LPM, for example about 30 LPM.

5. The flow rate control valve of any one of claims 1 to 4 wherein the second end is biased in the open position when the fluid flow rate is less than about 15 LPM.

6. The flow rate control valve of any one of claims 1 to 5 wherein the flexible closure is movable to a closed position when the fluid flow rate is greater than or equal to a closing flow rate, for example when the fluid flow rate is greater than about 60 LPM.

7. The flow rate control valve of claim 6 wherein the closed position substantially prevents fluid from flowing through the housing past the flexible closure.

8. The flow control valve of claim 6 or 7 wherein at least one of the flexible closure and the housing defines a by-pass aperture for fluid flow when the flexible closure is in the closed position, the by-pass aperture shaped and sized to limit the fluid flow rate to a predetermined flow rate.

9. The flow rate control valve of any one of claims 1 to 7 wherein the housing comprises an orifice plate defining an open area for fluid flow downstream of the flexible closure.

10. The flow rate control valve of claim 9 wherein the first end of the flexible closure abuts the orifice plate.

11. The flow rate control valve of claim 9 or 10 wherein the flexible closure is configured to seal against the orifice plate to substantially close the open area for fluid flow.

12. The flow rate control valve of any one of claims 9 to 11 wherein at least one of the orifice plate, the flexible closure and the housing defines a by-pass aperture for fluid flow when the flexible closure is in the closed position, the by-pass aperture shaped and sized to limit the fluid flow rate to a predetermined flow rate.

13. The flow rate control valve of any one of claims 9 to 12 wherein when the second end is biased in the open position, the second end is a distance from the orifice plate about equal to a thickness of the first end.

14. The flow rate control valve of any one of claims 1 to 13, further comprising a check valve disposed downstream of the flexible closure and upstream of a mouthpiece.

15. The flow rate control valve of any one of claims 1 to 14 wherein fluid flow is caused by inhalation.

16. The flow rate control valve of any one of claims 1 to 15 wherein the second end vibrates at a resonant frequency in the vibration position.

17. The flow rate control valve of any one of claims 1 to 16 wherein the vibrations of the flexible closure are sufficiently large to provide a haptic feedback to a user.

18. The flow rate control valve of any one of claims 1 to 17 wherein the flexible closure comprises two or more flexible closures.

19. The flow rate control valve of claim 18 wherein the two or more flexible closures vibrate at two or more different vibration inducing flowrates.

20. A flow rate control valve comprising,
a housing defining a cross-sectional area for fluid flow, and,
a flap connected to the housing and comprising a spring constant of from about 25 N/m to about 650 N/m,
wherein a fluid flow rate from about 15 LPM to about 60 LPM is sufficient to induce the flap to vibrate.

21. The flow rate control valve of claim 20 wherein the flap does not vibrate when the fluid flow is less than 15 LPM or greater than 60 LPM.

22. The flow rate control valve of claim 20 or 21 wherein the vibrations are sufficiently large to provide a haptic feedback to a user.

23. A device for delivering a medicated aerosol or mist to a subject, the device comprising the flow rate control valve according to any one of claims 1 to 22, wherein the device is connectable to an instrument for producing the aerosol or mist.

24. The device according to claim 23, further comprising a structure defining an aerosol- or mist-holding chamber upstream of the flow rate control valve.

25. The device according to claim 23, further comprising a structure defining an aerosol or mist-holding chamber downstream of the flow rate control valve.

26. The device according to claim 23, wherein the flow rate control valve is downstream of the instrument for producing the aerosol or mist.

27. The device according to claim 23, wherein the flow rate control valve is upstream of the instrument for producing the aerosol or mist.

28. The device according to claim 24 or 25 wherein the holding chamber comprises collapsible bellows.

29. The device according to claim 23 comprising a nasal occlusion face mask connected to the flow rate control valve.

30. The device according to claim 29 wherein the nasal occlusion face mask is adaptable to all facial anatomies.

31. A kit comprising the device according to any one of claims 23 to 30, and an instrument for producing the medicated aerosol or mist.

32. The device according to any one of claims 23 to 30, or the kit according to claim 31, wherein the medicated aerosol or mist is for the treatment or prevention of asthma or chronic obstructive pulmonary diseases.
